(19) United States
(12) Patent Application Publication
(10) Pub. No.: US 2006/0241729 A1
(43) Pub. Date: Oct. 26, 2006

(54) Method of treating nail fungus onychomycosis

(76) Inventor: Timothy Dawson, Dahlonega, GA (US)

Correspondence Address: TIM DAWSON, 564 WARD CREEK DR, DAHLONEGA, GA, 30533, US

(21) Appl. No.: 10/908,059

(22) Filed: Apr. 26, 2005

(51) Int. Cl. A61N 5/06

(52) U.S. Cl. 607/94

(57) ABSTRACT

A method of treating nail fungus is disclosed. The method comprises the application of ultraviolet light to an affected nail. Such nail treatment is selected from a reaction, a sterilizing reaction.

### BACKGROUND

Onychomycosis is a fungal infection of a nail, commonly a human nail. The fungus can invade the nail plate or nail bed, potentially causing the nail to separate from the nail bed, a thickening of the nail, discoloration, and other adverse symptoms. A convenient treatment for onychomycosis is needed that is both fast and safe for a care provider.

1. Field of the Invention

This invention relates to a method of treating nail fungus and more particularly to a method of utilizing a therapeutic ultraviolet light radiation by topical administration thereof to the affected nail.

2. Description of the Prior Art

Humans and animals alike are commonly plagued by the infiltration of micro-organisms beneath the nail, claw or hoof which result in a disease condition causing pain, discoloration, and frequently loss of the unguis. In humans, for example, some diseases which attack the nail or nail bed can be treated fairly successfully with the use of cleansing and/or antiseptic preparations, while other diseases require treatment by such means as systemic drug therapy. Nonetheless, many disease conditions of the nail, particularly onychomycosis (i.e., fungal diseases), have had a relatively low success rate for treatment due to the intransigent nature of the infectious micro-organisms.

It has been estimated that greater than twenty percent (20%) of the population of the United States over the age of 40 suffers from onychomycosis of the fingernails or toenails. The disease is known to occur to a lesser extent in people below the age of forty, but the occurrence of disease is still significant. Unfortunately, the current modalities for treatment of onychomycosis show a very low success rate.

Mycoses of the nails (onychomycosis) are intractable types of disease which have not been possible to treat satisfactorily. The term onychomycosis embraces various types of mycoses of the nails, of which those caused by dermatophytes are the most difficult to treat, whereas the mycoses of the nails caused by yeast-like fungi have hitherto been those which it has been possible most readily to treat successfully.

The difficulty with onychomycosis caused by dematophytes is additionally that they make a considerable contribution to the spread of infectious fungi. Various routes have been followed hitherto for their treatment, but without permanent success.

Common means of treating microbial diseases, including onychomycosis, include oral administration of drugs and laser therapy. Laser therapy, as yet, is not well-developed nor widely practiced, and is very expensive because it must be conducted in a doctor's office by a trained technician. Systematic drug therapy through oral administration has also proven to be relatively unsuccessful because of drug intolerances, the expense of the medications and low patient compliance. Typically, systemic oral treatment with antifungal agents requires long term treatment with a potential for toxic side effects.

A common means of treating onychomycosis is to remove the nail completely and topically apply medication to the underlying nail bed. However, not only is such treatment cosmetically unsightly, but the fungus which invades the nail often remains in the matrix of the finger or toe (where the nail is formed) and the disease reoccurs immediately upon, or during ingrowth of the new nail. Moreover, because of the aggressive nature of this treatment, the regrown nails are often deformed in shape.

A conservative method comprises topical treatment of the nails with specific substances having antimycotic activity. A very wide variety of treatment methods has been tried for this. Thus, in a combined treatment, the nails have initially been treated with solutions of the substances having antimycotic activity, and cream dressings have been applied each night. This treatment method is by its nature also very unpleasant for, and a psychological strain on, the patient. On the one hand, the nails have to be treated with a solution several times a day. On the other hand they have to be provided with dressings, particularly at night. Furthermore, it is necessary for the diseased nails to be filed or scraped down continually, which not only is troublesome but also contributes to spreading the pathogens. The results of all this are that many of the patients do not persist with the treatment, which usually takes several months; on the contrary, they become discouraged and negligent, and thus the therapy is unsuccessful. Also detrimental to the success of treatment by this method is that the solutions and creams are usually miscible with water or hydrophilic and thus can be removed again from the surface of the nail or dissolved out of the nail on washing, bathing and showering, and thus may need to be reapplied thereafter.

Undoubtedly, treatment of diseases involving nails would be greatly enhanced by the ability to access the area around and below the nail, as well as to penetrate the nail itself, without having to remove the nail. However, the thick and/or hardened nature of nails renders access through, and to the areas below, the nail very difficult. The same can be said of diseases involving the claws or hooves of animals.

The usefulness of antifungal drugs in treating onychomycosis has been limited heretofore because of resistance to penetration of the nail or because of limited access to the nail bed through the nail. Thus, it would be an improvement in the art to provide means for enhancing penetration of the nail so that treatment of the nail with ultraviolet light radiation may occur at the site and without having to remove or otherwise significantly damage the nail. It would also be advantageous to provide such penetration means at a reasonable cost to the consumer, and in a form which would facilitate and encourage proper and consistent self-use by the afflicted person.

### SUMMARY

This invention relates to a method of treating a nail fungus and, more particularly, to a method utilizing a therapeutic ultraviolet light radiation by topical administration thereof to the affected nail. The method comprises applying a therapeutic ultraviolet light on the nail.

A method of treating nail fungus is disclosed. The method comprises the application of ultraviolet light to an affected nail. Such nail treatment is selected from a reaction, a sterilizing reaction.

1. A method of treating onchomycosis of a nail of a Human and/or an animal, which comprises;
(a) an application of ultraviolet light to the exterior surface of the nail a therapeutically effective amount of a therapeutic ultraviolet light to sterilize thereon; and
(b) curing said area by altering the DNA of the said fungus on the nail bed.

2. The method as defined in claim 1 wherein said therapeutic application of ultraviolet light is selected from the group consisting of
(a) a sterilizing reaction;
(b) a stabilized reaction due to altered onychomycosis DNA;
(c) a blocked reaction of further growth of the fungus.

3. The method as defined in claim 2 1 wherein said reaction comprises an exposure of 50 to 5000 mJ/cm2 of full spectrum UV, wherein consist of 320-390 nm (UV-A) 280-320 nm (UV-B) 250-260 nm (UV-C) and 395-445 nm (UV-V).

4. The method as defined in claim 1 wherein said therapeutic UV exposure is comprised of short time exposures ranging from one second to multiple of minutes, which amounts to energy measured in joules/c2.

5. The method as defined in claim 1 wherein said ultraviolet light is selected from the group comprising primarily of UV-C 254 nm.

6. The method as defined in claim 1 wherein said stabilizing reaction is the results of the altered DNA of the fungus.

7. The method as defined in claim 1 wherein said altered DNA is rendered and the fungus is unable to reproduce.

8. The method as defined in claim 4 wherein said joules/cm2 (time) of exposure is determined by the severity of the onychomycosis.

9. (canceled)

10. The method as defined in claim 3 and 4 wherein said mJ/cm2 will be determined by the nail type and the severity of the onychomycosis being treated.
